(19) United States
(12) Patent Application Publication
(10) Pub. No.: US 2020/0206505 A1
(43) Pub. Date: Jul. 2, 2020

(54) Activation Code-Based Enablement of a Diagnostic Feature for a Cochlear Implant

(71) Applicant: Advanced Bionics AG, Staefa (CH)

(72) Inventors: Kurt J. Koester, Los Angeles, CA (US); Kanthaiah Koka, Valencia, CA (US); Leonid M. Litvak, Los Angeles, CA (US); Abhijit Kulkarni, Wellesley, MA (US)

(21) Appl. No.: 16/236,300

(22) Filed: Dec. 28, 2018

(51) Int. Cl. A61N 1/36; A61N 1/05

(52) U.S. Cl. CPC A61N 1/36039; A61N 1/08; A61N 1/0541

(57) ABSTRACT

An exemplary diagnostic system is configured to execute a diagnostic application, communicatively couple to a cochlear implant, receive, while communicatively coupled to the cochlear implant, input representative of an activation code, validate the activation code, link, in response to the validation, the activation code to a unique implant identifier associated with the cochlear implant, and enable, in response to the linking, a feature of the diagnostic application for use with the cochlear implant.

### BACKGROUND INFORMATION

During and after a surgical procedure in which a cochlear implant and an electrode lead are implanted within a recipient, it may be desirable to perform various diagnostic operations associated with the cochlear implant, electrode lead, and/or recipient. For example, during an insertion procedure in which an electrode lead is placed within the cochlea, it may be desirable to monitor evoked responses (e.g., electrocochleographic (“ECoG” or “ECochG”) potentials) that occur within the recipient in response to acoustic stimulation applied to the recipient. These evoked responses may be indicative of electrode positioning within the cochlea, trauma that may occur to the cochlea during the insertion procedure, residual hearing of different areas of the cochlea as the electrode lead is inserted, and/or various other factors associated with the insertion procedure.

To perform these diagnostic operations, a diagnostic system may execute a diagnostic application that has various features (e.g., testing capabilities, reporting capabilities, etc.). It may be desirable to control access to these features. For example, it may be desirable to ensure that features of the diagnostic application may always be used for a particular cochlear implant, regardless of the particular diagnostic system that is used to execute the diagnostic application. This may allow a recipient of the cochlear implant to switch between clinics without losing access to the features of the diagnostic application.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary cochlear implant system according to principles described herein.

FIG. 2 illustrates a schematic structure of the human cochlea according to principles described herein.

FIG. 3 illustrates an exemplary diagnostic system according to principles described herein.

FIG. 4 illustrates an exemplary stand-alone diagnostic system according to principles described herein.

FIG. 5 shows a base module detached from a computing module according to principles described herein.

FIGS. 6-8 depict exemplary configurations in which a diagnostic system is used to perform one or more diagnostic operations according to principles described herein.

FIGS. 9A-12 illustrate an exemplary hardware implementation of the diagnostic system of FIG. 4 according to principles described herein.

FIG. 13 illustrates an exemplary graphical user interface according to principles described herein.

FIG. 14 illustrates a disassembled surgical kit that may be provided for use during a procedure associated with a cochlear implant according to principles described herein.

FIG. 15 illustrates an exemplary graphical user interface according to principles described herein.

FIG. 16 illustrates various modules that may process input representative of an activation code and an implant identifier associated with a cochlear implant according to principles described herein.

FIG. 17 illustrates an exemplary configuration in which a diagnostic system is connected to a cloud-based management system by way of a network according to principles described herein.

FIG. 18 illustrates an exemplary method according to principles described herein.

FIG. 19 illustrates an exemplary computing device according to principles described herein.

### DETAILED DESCRIPTION

Systems and methods for activation-code based enablement of a diagnostic feature for a cochlear implant are described herein. For example, a diagnostic system may execute a diagnostic application, communicatively couple to a cochlear implant, and receive, while communicatively coupled to the cochlear implant, input representative of an activation code. The diagnostic system may validate the activation code and, in response, link the activation code to a unique implant identifier (“implant ID”) associated with the cochlear implant. In response to the linking of the activation code to the implant ID, the diagnostic system may enable a feature of the diagnostic application for use with the cochlear implant.

To illustrate, during a surgical procedure in which a cochlear implant and an electrode lead are implanted within a recipient, a user (e.g., a surgeon, assistant, etc.) may use a barcode scanner to scan a label included on packaging of a surgical kit that will be used during the surgical procedure. The label may include an activation code that is read by the barcode scanner. A diagnostic system (e.g., a handheld computing device) located in the operating room in which the surgical procedure takes place may receive the scanned activation code and attempt to validate the activation code. In response to determining that the activation code is valid, the diagnostic system may link the activation code to a unique implant ID associated with the cochlear implant. In response to this linking, the diagnostic system may enable one or more features of a diagnostic application being executed by the diagnostic system. For example, the diagnostic system may enable a feature that monitors an evoked response signal recorded by an electrode disposed on the electrode lead as the electrode lead is inserted into a cochlea of the recipient. In this manner, the diagnostic system may be used to facilitate correct placement of the electrode lead within the recipient.

In some examples, the systems and methods described herein are implemented by a stand-alone diagnostic system that includes a computing module and a base module configured to attach to the computing module (e.g., a back side of the computing module) and serve as a stand for the computing module. The computing module includes a display screen and a processor. The base module houses an interface unit configured to be communicatively coupled to the processor and to a cochlear implant while the base module is attached to the surface of the computing module. In this configuration, the processor may be configured to 1) execute a diagnostic application, 2) direct the display screen to display a graphical user interface associated with the diagnostic application, 3) query, by way of the interface unit, the cochlear implant for a unique implant ID associated with the cochlear implant, 4) receive, in response to the query, the unique implant ID from the cochlear implant, 5) receive, while the interface unit is communicatively coupled to the cochlear implant, input representative of an activation code, 6) validate the activation code, 7) link, in response to the validation, the activation code to the unique implant ID associated with the cochlear implant, 8) enable, in response to the linking, a feature of the diagnostic application for use with the cochlear implant, and 9) indicate, within the graphical user interface, that the feature is enabled for use with the cochlear implant.

Various advantages and benefits are associated with the systems and methods described herein. For example, the systems and methods described herein may improve an operation of a diagnostic system (e.g., a computing device that implements the diagnostic system) by specifically enabling various features of a diagnostic application executed by the diagnostic system for a particular cochlear implant.

The systems and methods described herein may additionally or alternatively allow a recipient of the cochlear implant to have access to a feature of the diagnostic application regardless of where the recipient goes to receive cochlear implant diagnostic services. For example, an activation code may be input into a diagnostic system used during an initial surgical operation in which a cochlear implant is implanted within a recipient. In response, the diagnostic system may enable a feature of the diagnostic system for use with the cochlear implant. The recipient may subsequently go to a different clinic (i.e., a clinic that did not perform the surgical operation) for a follow-up procedure (e.g., a fitting procedure). A different diagnostic system used by the different clinic may detect that an activation code has already been linked to an implant ID associated with the recipient's cochlear implant and therefore automatically enable the feature for use with the cochlear implant.

The systems and methods described herein may additionally or alternatively facilitate usage tracking of one or more features of a diagnostic application. For example, the systems and methods may allow a diagnostic system to keep a usage log of how many times a particular feature within a diagnostic application is used for a particular cochlear implant. The usage log may be used to perform analytics, notify a clinician of operations performed with respect to a particular recipient's cochlear implant during surgery, track usage for licensing purposes, etc.

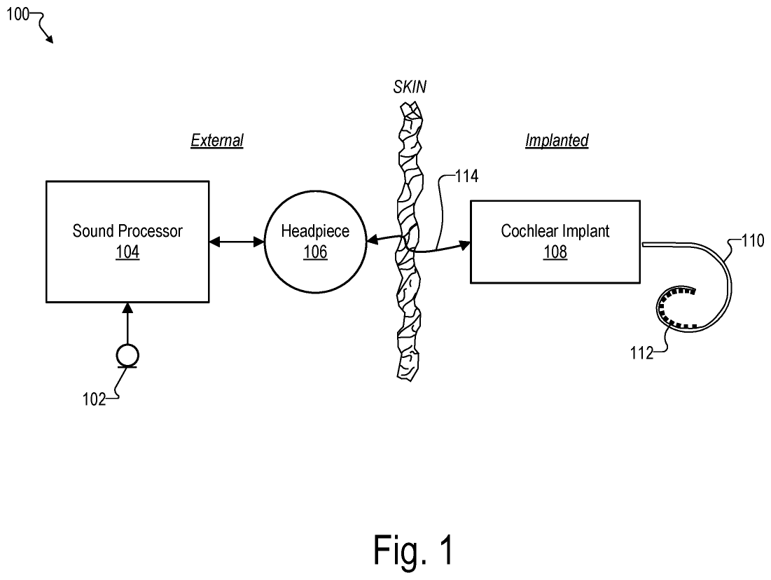

FIG. 1 illustrates an exemplary cochlear implant system 100. As shown, cochlear implant system 100 may include a microphone 102, a sound processor 104, a headpiece 106 having a coil disposed therein, a cochlear implant 108, and an electrode lead 110. Electrode lead 110 may include an array of electrodes 112 disposed on a distal portion of electrode lead 110 and that are configured to be inserted into a cochlea of a recipient to stimulate the cochlea when the distal portion of electrode lead 110 is inserted into the cochlea. One or more other electrodes (e.g., including a ground electrode, not explicitly shown) may also be disposed on other parts of electrode lead 110 (e.g., on a proximal portion of electrode lead 110) to, for example, provide a current return path for stimulation current generated by electrodes 112 and to remain external to the cochlea after electrode lead 110 is inserted into the cochlea. As shown, electrode lead 110 may be pre-curved so as to properly fit within the spiral shape of the cochlea. Additional or alternative components may be included within cochlear implant system 100 as may serve a particular implementation.

As shown, cochlear implant system 100 may include various components configured to be located external to a recipient including, but not limited to, microphone 102, sound processor 104, and headpiece 106. Cochlear implant system 100 may further include various components configured to be implanted within the recipient including, but not limited to, cochlear implant 108 and electrode lead 110.

Microphone 102 may be configured to detect audio signals presented to the user. Microphone 102 may be implemented in any suitable manner. For example, microphone 102 may include a microphone that is configured to be placed within the concha of the ear near the entrance to the ear canal, such as a T-MIC™ microphone from Advanced Bionics. Such a microphone may be held within the concha of the ear near the entrance of the ear canal during normal operation by a boom or stalk that is attached to an ear hook configured to be selectively attached to sound processor 104. Additionally or alternatively, microphone 102 may be implemented by one or more microphones disposed within headpiece 106, one or more microphones disposed within sound processor 104, one or more beam-forming microphones, and/or any other suitable microphone as may serve a particular implementation.

Sound processor 104 may be configured to direct cochlear implant 108 to generate and apply electrical stimulation (also referred to herein as “stimulation current”) representative of one or more audio signals (e.g., one or more audio signals detected by microphone 102, input by way of an auxiliary audio input port, input by way of a clinician's programming interface (CPI) device, etc.) to one or more stimulation sites associated with an auditory pathway (e.g., the auditory nerve) of the recipient. Exemplary stimulation sites include, but are not limited to, one or more locations within the cochlea, the cochlear nucleus, the inferior colliculus, and/or any other nuclei in the auditory pathway. To this end, sound processor 104 may process the one or more audio signals in accordance with a selected sound processing strategy or program to generate appropriate stimulation parameters for controlling cochlear implant 108. Sound processor 104 may be housed within any suitable housing (e.g., a behind-the-ear (“BTE”) unit, a body worn device, headpiece 106, and/or any other sound processing unit as may serve a particular implementation).

In some examples, sound processor 104 may wirelessly transmit stimulation parameters (e.g., in the form of data words included in a forward telemetry sequence) and/or power signals to cochlear implant 108 by way of a wireless communication link 114 between headpiece 106 and cochlear implant 108 (e.g., a wireless link between a coil disposed within headpiece 106 and a coil physically coupled to cochlear implant 108). It will be understood that communication link 114 may include a bi-directional communication link and/or one or more dedicated uni-directional communication links.

Headpiece 106 may be communicatively coupled to sound processor 104 and may include an external antenna (e.g., a coil and/or one or more wireless communication components) configured to facilitate selective wireless coupling of sound processor 104 to cochlear implant 108. Headpiece 106 may additionally or alternatively be used to selectively and wirelessly couple any other external device to cochlear implant 108. To this end, headpiece 106 may be configured to be affixed to the recipient's head and positioned such that the external antenna housed within headpiece 106 is communicatively coupled to a corresponding implantable antenna (which may also be implemented by a coil and/or one or more wireless communication components) included within or otherwise associated with cochlear implant 108. In this manner, stimulation parameters and/or power signals may be wirelessly transmitted between sound processor 104 and cochlear implant 108 via communication link 114.

Cochlear implant 108 may include any suitable type of implantable stimulator. For example, cochlear implant 108 may be implemented by an implantable cochlear stimulator. Additionally or alternatively, cochlear implant 108 may include a brainstem implant and/or any other type of cochlear implant that may be implanted within a recipient and configured to apply stimulation to one or more stimulation sites located along an auditory pathway of a recipient.

In some examples, cochlear implant 108 may be configured to generate electrical stimulation representative of an audio signal processed by sound processor 104 (e.g., an audio signal detected by microphone 102) in accordance with one or more stimulation parameters transmitted thereto by sound processor 104. Cochlear implant 108 may be further configured to apply the electrical stimulation to one or more stimulation sites (e.g., one or more intracochlear regions) within the recipient via electrodes 112 disposed along electrode lead 110. In some examples, cochlear implant 108 may include a plurality of independent current sources each associated with a channel defined by one or more of electrodes 112. In this manner, different stimulation current levels may be applied to multiple stimulation sites simultaneously by way of multiple electrodes 112.

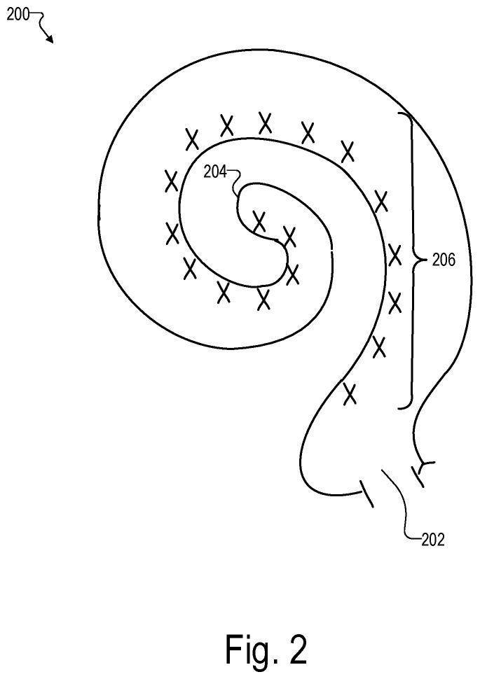

FIG. 2 illustrates a schematic structure of the human cochlea 200 into which electrode lead 110 may be inserted. As shown in FIG. 2, cochlea 200 is in the shape of a spiral beginning at a base 202 and ending at an apex 204. Within cochlea 200 resides auditory nerve tissue 206, which is denoted by Xs in FIG. 2. The auditory nerve tissue 206 is organized within the cochlea 200 in a tonotopic manner. Relatively low frequencies are encoded at or near the apex 204 of the cochlea 200 (referred to as an “apical region”) while relatively high frequencies are encoded at or near the base 202 (referred to as a “basal region”). Hence, electrical stimulation applied by way of electrodes disposed within the apical region (i.e., “apical electrodes”) may result in the recipient perceiving relatively low frequencies and electrical stimulation applied by way of electrodes disposed within the basal region (i.e., “basal electrodes”) may result in the recipient perceiving relatively high frequencies. The delineation between the apical and basal electrodes on a particular electrode lead may vary depending on the insertion depth of the electrode lead, the anatomy of the recipient's cochlea, and/or any other factor as may serve a particular implementation.

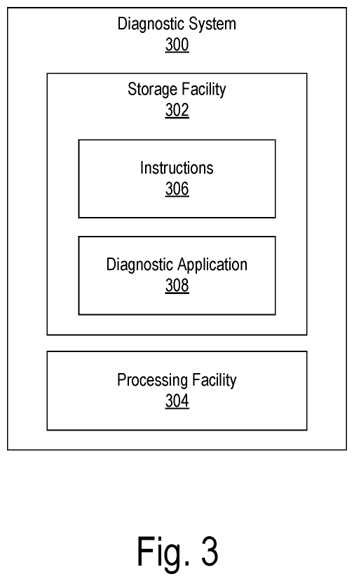

FIG. 3 illustrates an exemplary diagnostic system 300 that may be configured to perform any of the operations described herein. As shown, diagnostic system 300 may include, without limitation, a storage facility 302 and a processing facility 304 selectively and communicatively coupled to one another. Facilities 302 and 304 may each include or be implemented by hardware and/or software components (e.g., processors, memories, communication interfaces, instructions stored in memory for execution by the processors, etc.). In some examples, facilities 302 and 304 may be distributed between multiple devices and/or multiple locations as may serve a particular implementation.

Storage facility 302 may maintain (e.g., store) executable data used by processing facility 304 to perform any of the operations described herein. For example, storage facility 302 may store instructions 306 that may be executed by processing facility 304 to perform any of the operations described herein. Instructions 306 may be implemented by any suitable application, software, code, and/or other executable data instance.

As shown, storage facility 302 may also store data representative of a diagnostic application 308. Diagnostic application 308 may be executed by processing facility 304 to perform various diagnostic operations with respect to a cochlear implant before, during, and/or after a surgical procedure in which the cochlear implant is implanted within a recipient. Exemplary diagnostic operations are described herein. As described herein, diagnostic application 308 may include one or more features. These features may include, but are not limited to, various monitoring, measurement, interfacing, and reporting features.

Storage facility 302 may maintain additional or alternative data received, generated, managed, used, and/or transmitted by processing facility 304 as may serve a particular implementation.

Processing facility 304 may be configured to perform (e.g., execute instructions 306 stored in storage facility 302 to perform) various diagnostic operations. As one example, processing facility 304 may use diagnostic application 308 to monitor evoked responses that occur in response to acoustic stimulation applied during an insertion procedure in which an electrode lead connected to a cochlear implant is inserted into a cochlea of a recipient. The evoked responses may each be an ECoG potential (e.g., a cochlear microphonic potential, an action potential, a summating potential, etc.), an auditory nerve response, a brainstem response, a compound action potential, a stapedius reflex, and/or any other type of neural or physiological response that may occur within a recipient in response to application of acoustic stimulation to the recipient. Evoked responses may originate from neural tissues, hair cell to neural synapses, inner or outer hair cells, or other sources.

Evoked response monitoring and other exemplary diagnostic operations that may be performed by processing facility 304 in accordance with the systems and methods described herein are described in more detail in co-pending PCT Application No. PCT/US18/67900, Attorney Docket No. 3021-0493-WO, co-pending PCT application Ser. No. ______, Attorney Docket No. 3021-0494-WO, co-pending PCT application Ser. No. ______, Attorney Docket No. 3021-0495-WO, and co-pending U.S. application Ser. No. ______, Attorney Docket No. 3021-0497, each of which is filed the same day as the present application and incorporated herein by reference in its entirety.

Diagnostic system 300 may be implemented in any suitable manner. For example, diagnostic system 300 may be implemented by a stand-alone diagnostic system that may be used in a surgical operating room to perform any of the operations described herein.

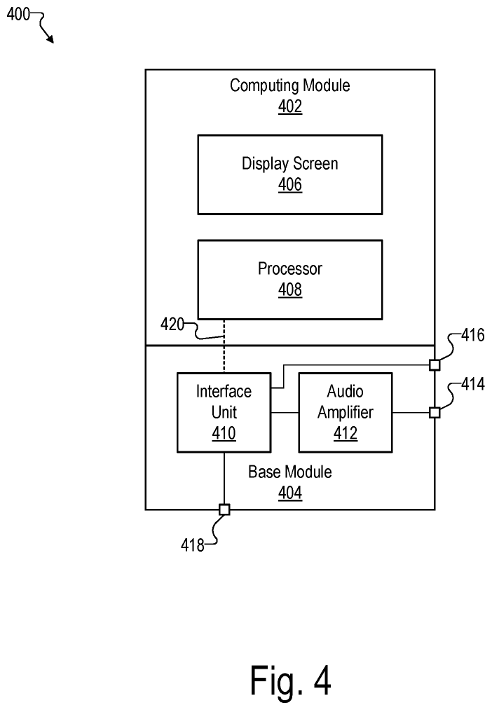

FIG. 4 illustrates an exemplary stand-alone diagnostic system 400 that may implement diagnostic system 300. As shown, diagnostic system 400 includes a computing module 402 and a base module 404. Computing module 402 includes a display screen 406 and a processor 408. Base module 404 includes an interface unit 410, an audio amplifier 412, an audio output port 414, a communications port 416, and a port 418. Computing module 402 and base module 404 may include additional or alternative components as may serve a particular implementation. For example, computing module 402 and/or base module 404 may include one or more speakers configured to output acoustic feedback and/or other types of sound configured to be heard by a surgeon and/or other user of diagnostic system 400. Diagnostic system 400 and exemplary implementations thereof are described more fully in co-pending PCT Application No. PCT/US18/67900, which application is filed the same day as the present application and incorporated herein by reference in its entirety.

In the configuration shown in FIG. 4, base module 404 is physically attached to computing module 402. In this configuration, processor 408 is communicatively coupled to interface unit 410 by way of a connection 420. Connection 420 may be implemented by any suitable connection (e.g., an internal USB connection) as may serve a particular implementation. As will be described in more detail below, base module 404 may be selectively detached from computing module 402 and connected to a different computing device by way of port 418.

Display screen 406 may be configured to display any suitable content associated with an application executed by processor 408. Display screen 406 may be implemented by a touchscreen and/or any other type of display screen as may serve a particular implementation.

Processor 408 may be configured to execute a diagnostic application (e.g., diagnostic application 308) associated with a cochlear implant (e.g., cochlear implant 108). For example, processor 408 may execute a diagnostic application that may be used before, during, or after a surgical procedure associated with the cochlear implant. The diagnostic application may be used by processor 408 to perform various diagnostic operations with respect to the cochlear implant during the surgical procedure. Exemplary diagnostic operations are described herein.

In some examples, processor 408 may direct display screen 406 to display a graphical user interface associated with the diagnostic application being executed by processor 408. A user may interact with the graphical user interface to adjust one or more parameters associated with the cochlear implant and/or otherwise obtain information that may be useful during a procedure associated with the cochlear implant.

Base module 404 may be configured to attach to computing module 402 and serve as a stand for computing module 402.

Interface unit 410 is configured to be communicatively coupled to processor 408 by way of connection 420 while base module 404 is attached to computing module 402. Interface unit 410 is further configured to be communicatively coupled to the cochlear implant while base module 404 is attached to the computing module 402. In this manner, interface unit 410 provides an interface between processor 408 and the cochlear implant.

Interface unit 410 may be communicatively coupled to the cochlear implant by way of communications port 416. For example, communications port 416 may be selectively coupled to a coil (e.g., a coil included in a headpiece, such as headpiece 106, or a disposable stand-alone coil) configured to wirelessly communicate with the cochlear implant. Interface unit 410 may communicate with the cochlear implant by transmitting and/or receiving data to/from the cochlear implant by way of the coil connected to communications port 416.

Interface unit 410 may be further configured to generate and provide acoustic stimulation (e.g., sound waves) to the recipient of the cochlear implant. To this end, audio output port 414 is configured to be selectively coupled to a sound delivery apparatus. In some examples, the sound delivery apparatus may be implemented by tubing that has a distal portion configured to be placed in or near an entrance to an ear canal of a recipient of the cochlear implant. While the sound delivery apparatus is connected to audio output port 414, interface unit 410 may transmit the acoustic stimulation to the recipient by way of the sound delivery apparatus.

As shown, audio amplifier 412 may be positioned within a path between interface unit 410 and audio output port 414. In this configuration, audio amplifier 412 may be configured to amplify the acoustic stimulation before the acoustic stimulation is delivered to the recipient by way of audio output port 414 and the sound delivery apparatus. In some alternative examples, amplification of the acoustic stimulation generated by interface unit 410 is not necessary, thereby obviating the need for audio amplifier 412 to be included in base module 404. Hence, in some implementations, base module 404 does not include audio amplifier 412.

In some examples, diagnostic system 400 may be configured to self-calibrate and/or perform in-situ testing. For example, processor 408 may calibrate an amplitude level of acoustic stimulation generated by interface unit 410 before and/or during a surgical procedure. Such self-calibration and in-situ testing may be performed in any suitable manner.

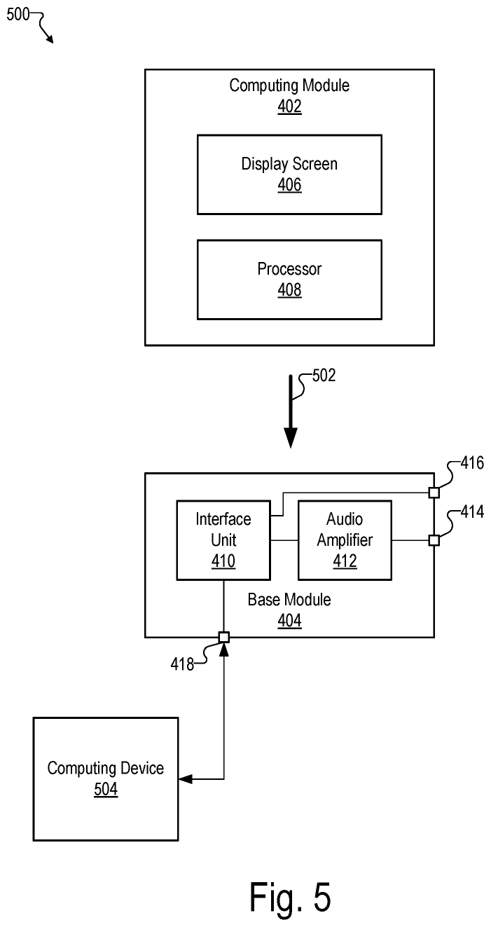

As mentioned, base module 404 may be selectively detached from computing module 402. To illustrate, FIG. 5 shows a configuration 500 in which base module 404 is detached from computing module 402. This detachment is illustrated by arrow 502. While detached, interface unit 410 of base module 404 may be communicatively coupled to a computing device 504. For example, interface unit 410 may be communicatively coupled to computing device 504 by plugging a cable (e.g., a USB cable) into port 418 and into computing device 504. In this configuration, computing device 504 may use interface unit 410 to interface with a cochlear implant (e.g., by providing acoustic stimulation to a recipient of the cochlear implant and/or receiving recording data from the cochlear implant).

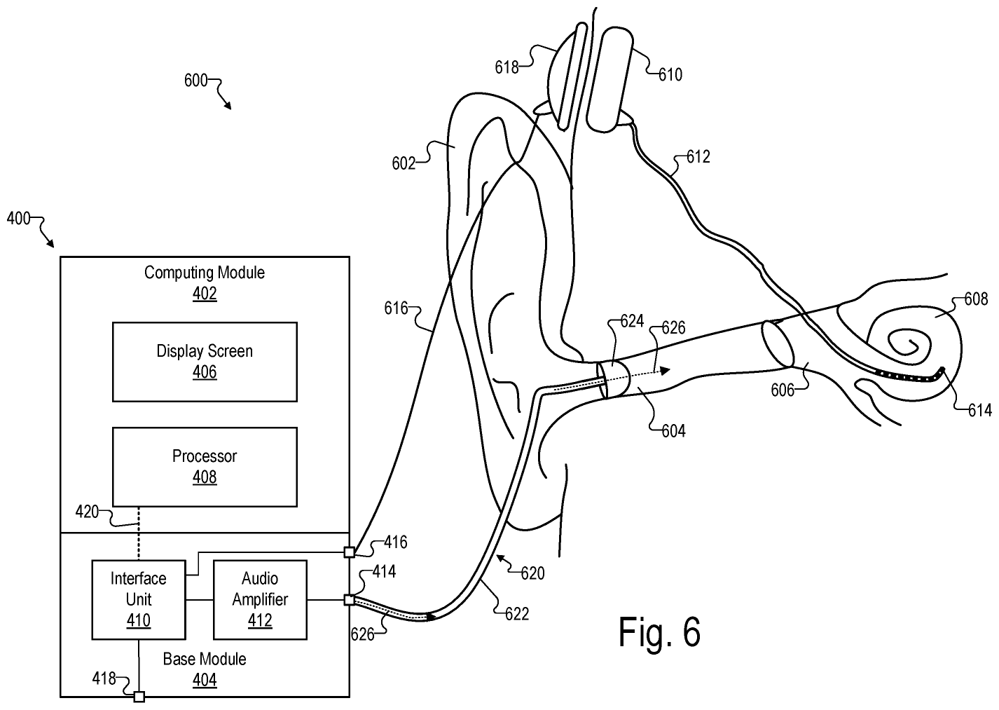

FIG. 6 depicts an exemplary configuration 600 in which diagnostic system 400 is used to perform one or more diagnostic operations during a surgical procedure involving a cochlear implant and an electrode lead. The surgical procedure may include, for example, an insertion procedure in which the cochlear implant is inserted into an incision pocket formed within the recipient and/or in which a distal portion of the electrode lead is positioned within the cochlea.

Various anatomical features of the recipient's ear are shown in FIG. 6. Specifically, anatomical features include a pinna 602 (i.e., the outer ear), an ear canal 604, a middle ear 606, and a cochlea 608. While no specific incision or other explicit surgical representation is shown in FIG. 6, it will be understood that such elements may be present when a surgical procedure is ongoing. For example, an incision may be present to allow the surgeon internal access to the recipient to insert the lead into cochlea 608. In some procedures, pinna 602 may be taped down and covered with surgical drapes so as to cover ear canal 604 (e.g., to help prevent fluids from reaching ear canal 604).

In the example of FIG. 6, a cochlear implant 610 and an electrode lead 612 are shown to be implanted within the recipient. Cochlear implant 610 may be similar, for example, to cochlear implant 108, and electrode lead 612 may be similar, for example, to electrode lead 110. Electrode lead 612 includes a plurality of electrodes (e.g., electrode 614, which is the distal-most electrode disposed on electrode lead 612).

As shown, a cable 616 of a headpiece 618 is connected to communications port 416. In this configuration, interface unit 410 may wirelessly communicate with cochlear implant 610 by way of a coil and/or other electronics included in headpiece 618, which may be similar to headpiece 106.

As also shown, a sound delivery apparatus 620 is connected to audio output port 414. Sound delivery apparatus 620 includes tubing 622 and an ear insert 624. Ear insert 624 is configured to fit at or within an entrance of ear canal 604. Tubing 622 and ear insert 624 together form a sound propagation channel 626 that delivers acoustic stimulation provided by interface unit 410 to the ear canal 604. Tubing 622 and ear insert 624 may be made out of any suitable material as may serve a particular implementation.

In some examples, processor 408 may execute a diagnostic application during the surgical procedure. In accordance with the diagnostic application, processor 408 may transmit, by way of connection 420, a command (also referred to as a stimulation command) to interface unit 410 for interface unit 410 to apply acoustic stimulation to the recipient and receive recording data representative of an evoked response that occurs within the recipient in response to the acoustic stimulation. In response to receiving the command, interface unit 410 may generate and apply the acoustic stimulation to the recipient by way of audio output port 414 and sound delivery apparatus 620. Interface unit 410 may also transmit a command (also referred to as a recording command) to cochlear implant 610 by way of communications port 416 and headpiece 618 for cochlear implant 610 to use electrode 614 to record the evoked response that occurs in response to the acoustic stimulation. Cochlear implant 610 may transmit the recording data back to interface unit 410 by way of headpiece 618 and communications port 416. Interface unit 410 may transmit the recording data to processor 408 by way of connection 420. Processor 408 may process the recording data and direct display screen 406 to display one or more graphical user interfaces associated with the recording data.

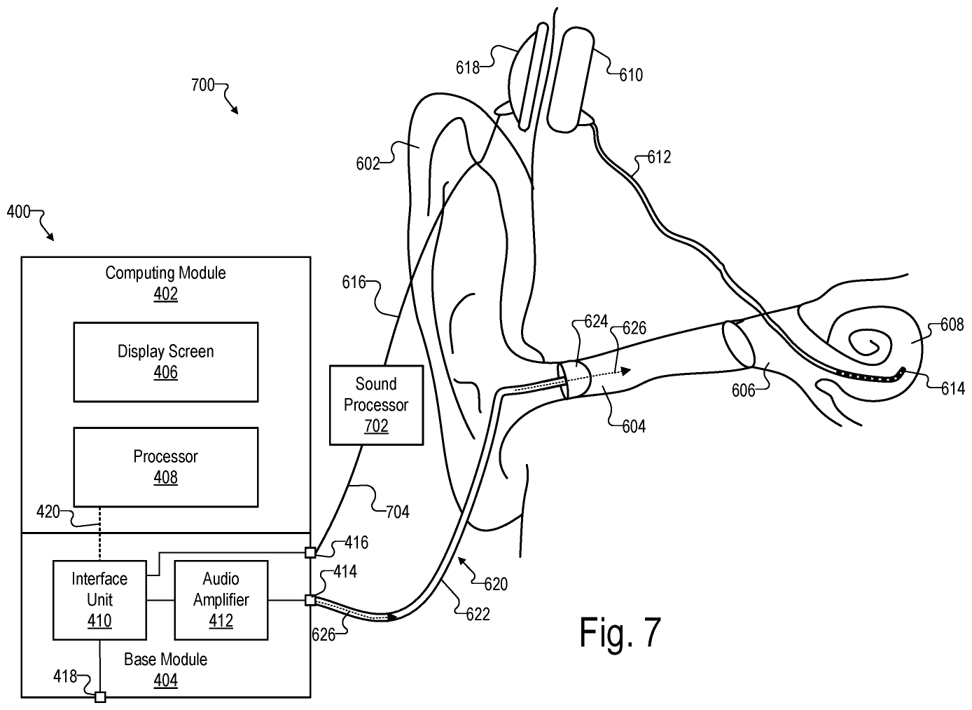

In configuration 600, headpiece 618 is connected directly to communications port 416 by way of cable 616. Hence, in configuration 600, interface unit 410 is configured to directly control cochlear implant 610. FIG. 7 illustrates an alternative configuration 700 in which a sound processor 702 is included in the communication path in between interface unit 410 and cochlear implant 610. Sound processor 702 may be similar to any of the sound processors (e.g., sound processor 104) described herein. In some examples, sound processor 702 is recipient-agnostic. In other words, sound processor 702 is not configured specifically for the recipient of cochlear implant 610. Rather, sound processor 702 may be used in a variety of different surgical procedures associated with a number of different recipients.

As shown, sound processor 702 is connected to communications port 416 by way of a cable 704. Sound processor 702 is also connected to headpiece 618 by way of cable 616. In this configuration, sound processor 702 may relay data and/or commands between interface unit 410 and cochlear implant 610.

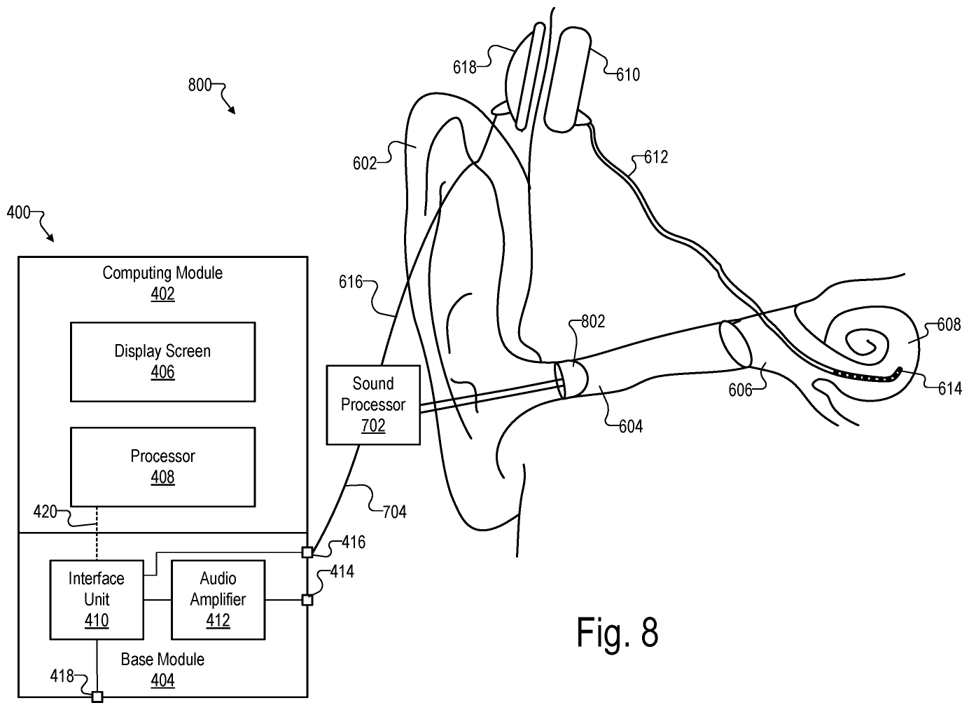

FIG. 8 illustrates an alternative configuration 800 in which sound processor 702 is configured to generate the acoustic stimulation that is applied to the recipient of cochlear implant 610. As shown, in this configuration, a sound delivery apparatus 802 is coupled directly to sound processor 702. For example, sound processor 702 may be implemented by a behind-the-ear bimodal sound processor and sound delivery apparatus 802 may be implemented by an audio ear hook that connects to sound processor 702.

It will be recognized that diagnostic system 400 may be additionally or alternatively implemented in any other suitable manner. For example, diagnostic system 400 may be implemented by a fitting system utilized in a clinician's office and/or by any other appropriately configured system or device.

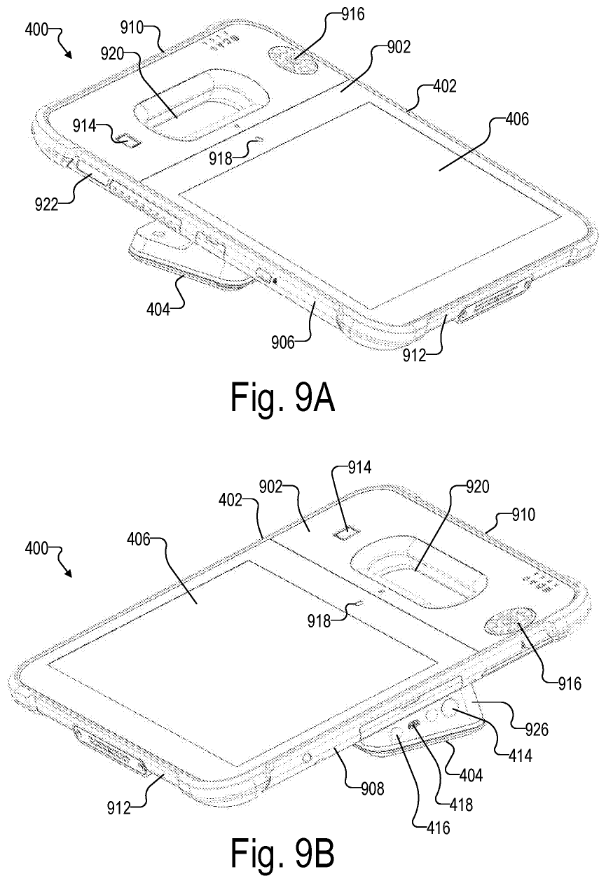

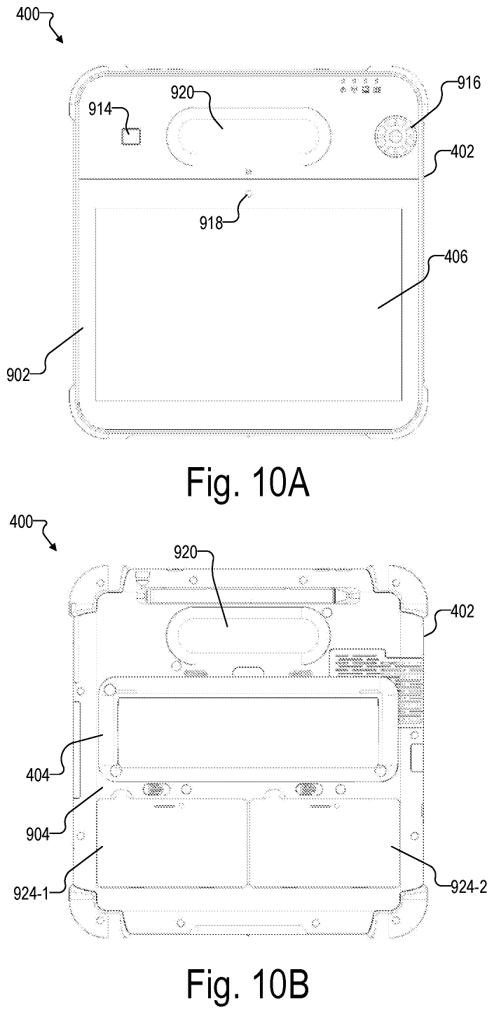

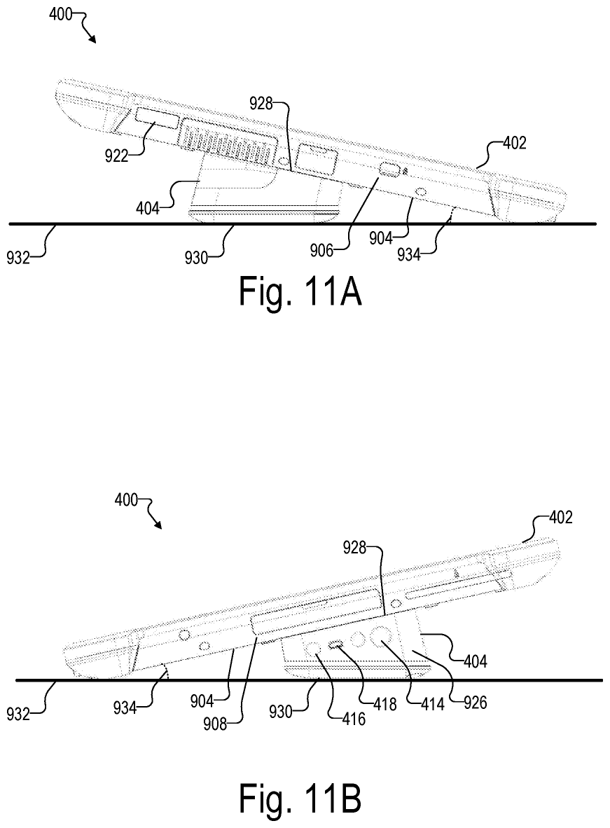

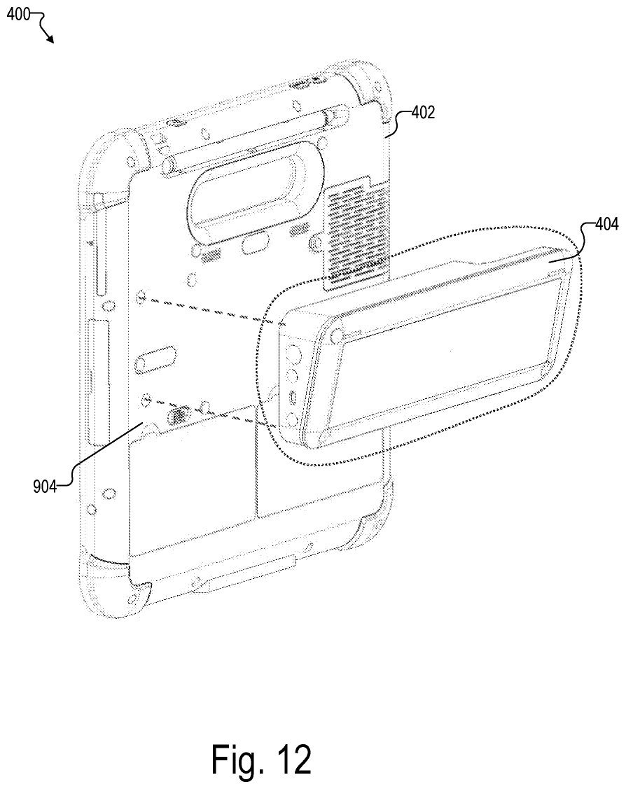

An exemplary hardware implementation of diagnostic system 400 will now be described in connection with FIGS. 9A-12. In particular, FIG. 9A shows a left perspective view of diagnostic system 400, FIG. 9B shows a right perspective view of diagnostic system 400, FIG. 10A shows a front view of diagnostic system 400, FIG. 10B shows a back view of diagnostic system 400, FIG. 11A shows a left side view of diagnostic system 400, FIG. 11B shows a right side view of diagnostic system 400, and FIG. 12 shows a rear perspective view of diagnostic system 400.

The hardware implementation of diagnostic system 400 illustrated in FIGS. 9A-12 includes computing module 402 and base module 404. As, illustrated computing module 402 includes a front side 902, a back side 904, a left side 906, a right side 908, a top side 910, and a bottom side 912.

Display screen 406 is located on front side 902 of computing module 402. Various other components are also located on the front side 902 of computing module 402. For example, a fingerprint scanner 914, physical input buttons 916, and a webcam 918 all shown to be included on the front side 902 of computing module 402. It will be recognized that any of these components may be located on any other side of computing module 402 as may serve a particular implementation.

Fingerprint scanner 914 is configured to facilitate authentication of a user of diagnostic system 400. For example, fingerprint scanner 914 may detect a fingerprint of the user and provide processor 408 with data representative of the fingerprint. Processor 408 may process the fingerprint data in any suitable manner (e.g., by comparing the fingerprint to known fingerprints included in a database) to authenticate the user.

Webcam 918 may be configured to facilitate video communication by a user of diagnostic system 400 with a remotely located user (e.g., during a surgical procedure). Such video communication may be performed in any suitable manner.

Physical input buttons 916 may be implemented, for example, by a directional pad and/or any other suitable type of physical input button. A user of diagnostic system 400 may interact with physical input buttons 916 to perform various operations with respect to a diagnostic application being executed by processor 408. For example, the user may use the physical input buttons 916 to interact with a graphical user interface displayed on display screen 406.

In some examples, physical input buttons 916 may be configured to be selectively programmed (e.g., as hotkeys) to perform one or more functions associated with the diagnostic application. For example, a particular physical input button 916 may be programmed by a user to start and/or stop acoustic stimulation being applied to a cochlear implant recipient by diagnostic system 400.

In some examples, processor 408 may be configured to wirelessly connect to an input device configured to be used by the user in connection with the diagnostic application. For example, processor 408 may be configured to wirelessly connect (e.g., via Bluetooth and/or any other suitable wireless communication protocol) to a keyboard, mouse, remote control, and/or any other wireless input device as may serve a particular implementation. In this manner, the user may selectively use physical input buttons 916, a touchscreen capability of display screen 406, and/or a wireless input device to interact with diagnostic system 400.

As shown, a hole 920 may be formed within computing module 402 and configured to serve as a handle for diagnostic system 400. A user may grip computing module 402 by placing his or her fingers within hole 920.

As shown, a barcode scanner 922 may be located on left side 906 of computing module 402. Barcode scanner 922 may alternatively be located on any other side of computing module 402. In some examples, barcode scanner 922 may be configured to scan for an activation code included on one or more components associated with a procedure being performed with respect to cochlear implant 510. An example of this will be provided herein.

As illustrated in FIG. 10B, computing module 402 may include batteries 924-1 and 924-2. Batteries 924 may be configured to provide operating power for various components included within computing module 402 and base module 404. In some examples, batteries 924 may be hot-swappable. In other words, one of batteries 924 (e.g., battery 924-1) may be removed and replaced while the other battery (e.g., battery 924-2) is used to provide power to computing module 402 and base module 404.

As illustrated in FIGS. 9B and 11B, ports 414, 416, and 418 are located on a side surface 926 of base module 404. Ports 414, 416, and 418 may alternatively be located on any other surface of base module 404.

As described above, base module 404 may be configured to serve as a stand for computing module 402 while base module 404 is attached to computing module 402. The stand functionality of base module 404 is illustrated in FIGS. 11A-11B.

As shown, base module 404 includes a top surface 928 configured to selectively attach to back side 904 of computing module 402. Base module 404 may alternatively attach to any other side of computing module 402. Base module 404 further includes a bottom surface 930 configured to be placed on a resting surface 932. Bottom surface 930 is angled with respect to back side 904 of computing module 402. This provides a viewing angle 934 for display screen 406 that is greater than zero degrees with respect to resting surface 932. In some examples, base module 404 may be adjustable to selectively provide different viewing angles for display screen 406 with respect to resting surface 932. This adjustability may be realized in any suitable manner. For example, a user may manually adjust bottom surface 930 to different angles with respect to back side 904 of computing module 402.

FIG. 12 illustrates an exemplary configuration in which base module 404 is detached from computing module 402. Base module 404 may be detached from computing module 402 in any suitable manner. For example, base module 404 may include one or more locking mechanisms that may be actuated by a user to detach base module 404 from computing module 402.

Various operations that may be performed by diagnostic system 300 will now be described. It will be recognized that diagnostic system 300 may perform additional or alternative operations to those described herein as may serve a particular implementation.

As mentioned, diagnostic system 300 may communicatively couple to a cochlear implant. This may be performed in any of the ways described herein. For example, diagnostic system 300 may establish a connection with the cochlear implant by way of a cable that connects diagnostic system 300 to a sound processor that is wirelessly connected to the cochlear implant by way of a coil included in a headpiece.

While communicatively coupled to the cochlear implant, diagnostic system 300 may execute a diagnostic application. This may be performed in any of the ways described herein. In some examples, as part of executing the diagnostic application, diagnostic system 300 may present (e.g., direct a display device to display) a graphical user interface associated with the diagnostic application.

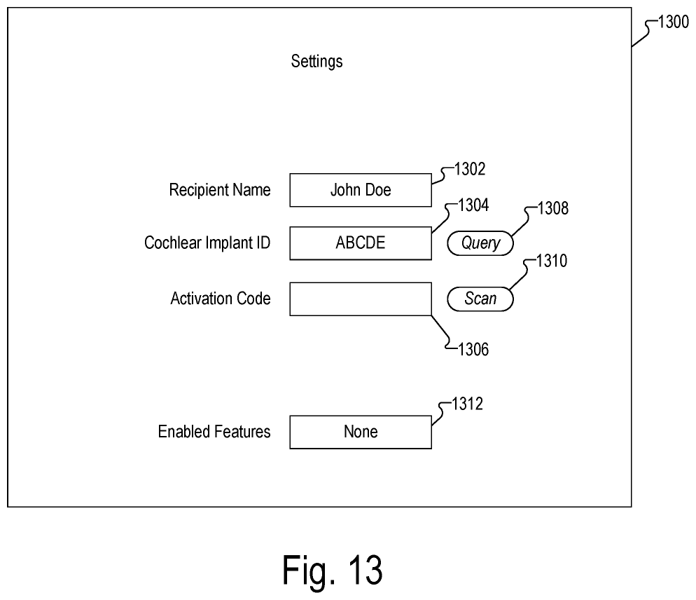

FIG. 13 shows an exemplary graphical user interface 1300 that may be presented by diagnostic system 300. Graphical user interface 1300 may be used to provide various types of information used by the diagnostic application. For example, a name of a recipient of a cochlear implant may be entered into field 1302, an implant ID associated with the cochlear implant may be entered into field 1304, and an activation code may be entered into field 1306.

In some examples, the implant ID entered into field 1304 is unique to the particular cochlear implant to which diagnostic system 300 is communicatively coupled. For example, the implant ID may include a serial number or other unique identifier of the cochlear implant. In some examples, data representative of the implant ID is stored permanently in the cochlear implant. In the particular example of FIG. 13, the implant ID of the cochlear implant communicatively coupled to diagnostic system 300 is ABCDE.

Input representative of the implant ID may be provided to diagnostic system 300 in any suitable manner. For example, the implant ID may be entered manually into field 1304. Alternatively, a user may select a query option 1308 to direct diagnostic system 300 to query the cochlear implant for the implant ID. In response to the selection of query option 1308, diagnostic system 300 may query the cochlear implant for the implant ID by transmitting a command to the cochlear implant (e.g., by way of a sound processor and a coil included in a headpiece) for the cochlear implant to provide diagnostic system 300 with data representative of the implant ID. In response to receiving the command, the cochlear implant may transmit the requested data to diagnostic system 300, which may process the data and automatically populate field 1304 with the implant ID. In some alternative examples, diagnostic system 300 may be configured to automatically query the cochlear implant for the implant ID in response to communicatively coupling to the cochlear implant. In this manner, the user may not have to manually enter the implant ID or select the query option 1308 in order to provide the implant ID to diagnostic system 300.

The activation code may be provided to diagnostic system 300 in any suitable manner. For example, the activation code may be entered manually into field 1306. Alternatively, a user may select a scan option 1310 to use a barcode scanner (e.g., barcode scanner 922) of diagnostic system 300 to provide the activation code to diagnostic system 300. In some examples, the user may simply use the barcode scanner to provide the activation code to diagnostic system 300 without first selecting scan option 1310.

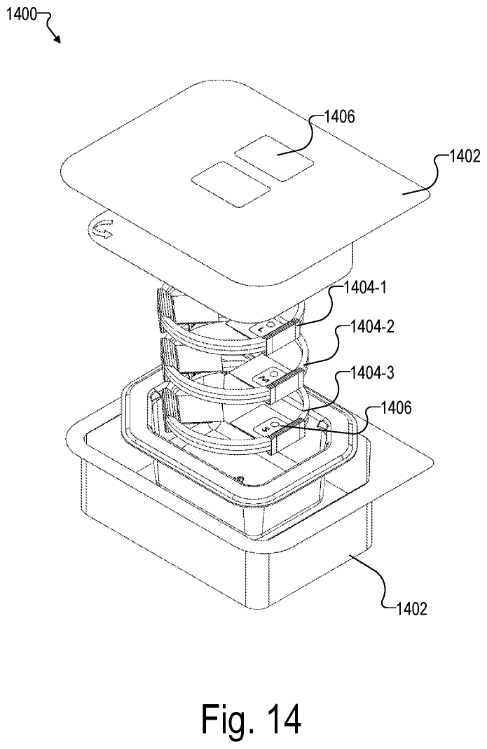

To illustrate the use of the barcode scanner, FIG. 14 illustrates a disassembled surgical kit 1400 that may be provided for use in conjunction with diagnostic system 300 during a procedure associated with a cochlear implant. Surgical kit 1400 includes packaging 1402 and a plurality of sound delivery apparatuses 1404 (e.g., sound delivery apparatus 1404-1 through sound delivery apparatus 1404-3). One or more other components may be included in surgical kit 1400 as may serve a particular implementation.

As shown, packaging 1402 may include an activation code label 1406. Activation code label 1406 may be scanned by barcode scanner 922 to provide diagnostic system 300 with an activation code. In response to receiving the activation code by way of barcode scanner 922, diagnostic system 300 may populate field 1306 with the received activation code. It will be recognized that an activation code may be included in any other type of scannable object as may serve a particular implementation.

Additionally or alternatively, diagnostic system 300 may query the cochlear implant for the activation code. For example, in some instances, a manufacturer of a cochlear implant may load data representative of the activation code into memory of a cochlear implant before the cochlear implant is provided to an end user. In these instances, diagnostic system 300 may receive data representative of the activation code directly from the cochlear implant.

Returning to the particular example of FIG. 13, an activation code has not yet been input into field 1306. Hence, diagnostic system 300 indicates in field 1312 that no features of the diagnostic application are currently enabled for the cochlear implant having the cochlear implant ID of ABCDE.

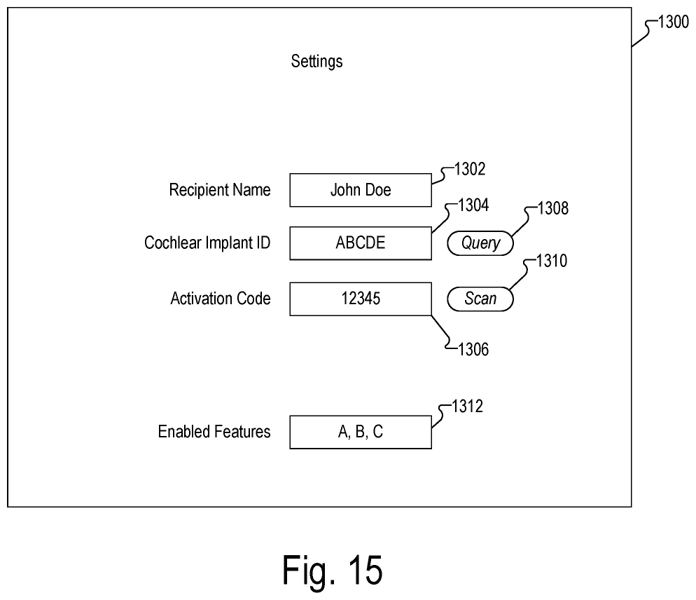

FIG. 15 shows graphical user interface 1300 after an activation code of 12345 has been entered into field 1306. In response to receiving the activation code, diagnostic system 300 may attempt to validate the activation code. Examples of this will be provided herein. If diagnostic system 300 determines that the activation code is valid, diagnostic system 300 may enable one or more features of the diagnostic application for use with the cochlear implant having the cochlear implant ID shown in field 1304. In the particular example of FIG. 15, diagnostic system 300 determined that the activation code 12345 shown in field 1306 is valid. Hence, field 1312 shows that features A, B, and C are enabled for use with the cochlear implant having the implant ID shown in field 1304. Diagnostic system 300 may indicate within graphical user interface 300 that one or more features of the diagnostic application are enabled for use with the cochlear implant in any other suitable manner.

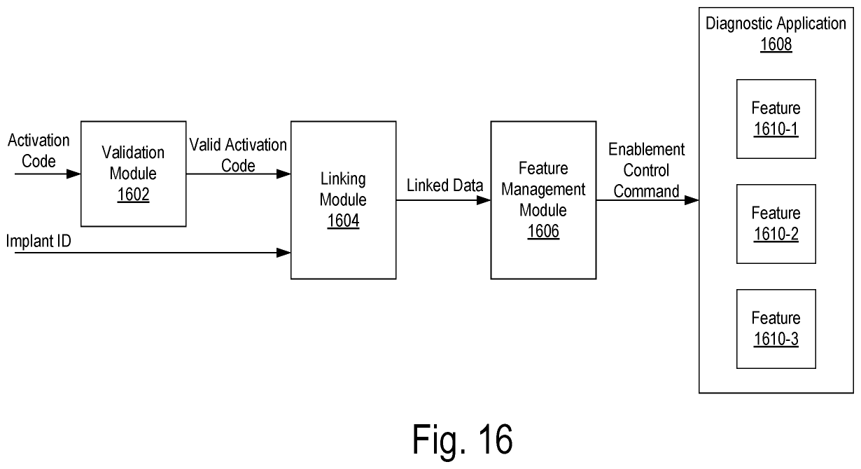

FIG. 16 illustrates various modules 1602-1606 that may be implemented by diagnostic system 300 (e.g., by processing facility 304) and that may process input representative of an activation code and an implant ID associated with a particular cochlear implant. In particular, the modules illustrated in FIG. 16 illustrate how the activation code and the implant ID may be used to selectively enable one or more features of a diagnostic application 1608 executed by diagnostic system 300.

As shown, data representative of an activation code is input into a validation module 1602. Validation module 1602 is configured to determine whether the activation code is valid. This validation may be performed in any suitable manner.

For example, validation module 1602 may validate the activation code by determining that the activation code has one or more valid characteristics. For example, validation module 1602 may determine whether a length, size, character type, etc. matches predetermined values indicative of a valid activation code. As another example, validation module 1602 may generate a hash value associated with the activation code and determine whether the hash value is valid in any suitable manner.

As another example, validation module 1602 may validate the activation code by determining whether the activation code is included in a database of valid activation codes. The database may be stored locally by diagnostic system 300, remotely by a server, and/or at any other suitable location. Validation module 1602 may access the database in any suitable manner.

If validation module 1602 determines that the activation code is valid, validation module 1602 may transmit data representative of the valid activation code to a linking module 1604. If validation module 1602 determines that the activation code is invalid, validation module 1602 may either abstain from transmitting data to linking module 1604 or transmit data that indicates that the activation code is invalid to linking module 1604. In either case, the invalid activation code will result in features of diagnostic application 1608 being disabled. For purposes of this example, it will be assumed that validation module 1602 determines that the activation code is valid.

As shown, linking module 1604 also receives an implant ID of the cochlear implant as an input. Linking module 1604 is configured to link the activation code to the implant ID. This may be performed in any suitable manner.

For example, linking module 1604 may link the activation code to the implant ID by associating the activation code with the implant ID within a database (e.g., within a database stored within storage facility 302 and/or any other suitable storage location). To illustrate, linking module 1604 may store the activation code and the implant ID within a lookup table in a manner that indicates that the activation code is associated with the implant ID.

As shown, linking module 1604 may provide linked data (i.e., data representative of the link between the activation code and the implant ID) to a feature management module 1606. Feature management module 1606 may use the linked data to generate an enablement control command, which may be provided to diagnostic application 1608 to selectively enable one or more features 1610 of diagnostic application 1608. In the example of FIG. 16, diagnostic application 1608 includes three features 1610-1 through 1610-3. However it will be recognized that diagnostic application 1608 may alternatively include any number of features as may serve a particular implementation.

In some examples, feature management module 1606 may enable all of features 1610 of diagnostic application 1608 based on a valid activation code being received by validation module 1602. Alternatively, feature management module 1606 may selectively enable only a subset of features 1610 of diagnostic application 1608 based on a valid activation code being received by validation module 1602. For example, a particular activation code may have a particular characteristic (e.g., be within a certain range of values) that indicates that only a particular feature (e.g., feature 1610-1) is to be enabled. In this example, feature management module 1606 may enable feature 1610-1 while keeping features 1610-2 and 1610-3 disabled.

Diagnostic system 300 may store data representative of the linking of the activation code to the implant ID in any suitable manner. For example, diagnostic system 300 may locally store this data within a storage device (e.g., storage facility 302) of diagnostic system 300. In this manner, when the cochlear implant is subsequently connected again to diagnostic system 300, diagnostic system 300 may recognize that a valid activation code is already linked to the implant ID of the cochlear implant and automatically enable one or more features of the diagnostic application without requiring an activation code to again be provided for the cochlear implant.

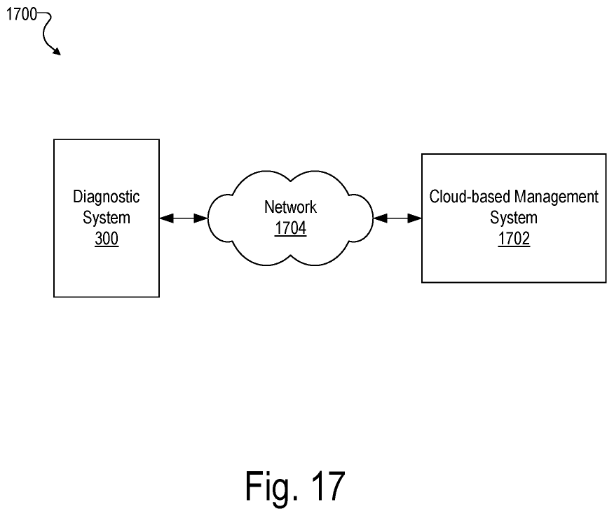

Additionally or alternatively, diagnostic system 300 may transmit data representative of the linking of the activation code to the implant ID to a remote computing device for remote storage by the computing device. For example, FIG. 17 illustrates an exemplary configuration 1700 in which diagnostic system 300 is connected to a cloud-based management system 1702 by way of a network 1704.

Network 1704 may be implemented by any suitable network, such as the Internet, a wide area network, a local area network, a provider-specific wired or wireless network (e.g., a cable or satellite carrier network or a mobile telephone network), a content delivery network, and/or any other suitable network. Data may flow between diagnostic system 300 and cloud-based management system 1702 using any communication technologies, devices, media, and protocols as may serve a particular implementation.

Cloud-based management system 1702 may be implemented by one or more server-side computing devices configured to communicate with diagnostic system 300 by way of network 1704. For example, cloud-based management system 1702 may be implemented by one or more servers or other physical computing devices.

In some examples, cloud-based management system 1702 may store activation code data for a plurality of cochlear implants. In this manner, diagnostic system 300 and/or any other diagnostic system (e.g., a different diagnostic system located at a different clinic or other location) may communicate with cloud-based management system 1702 to determine whether a particular cochlear implant has already been associated with a valid activation code. This may allow a recipient of a cochlear implant to have access to the diagnostic application regardless of where recipient goes to receive diagnostic services as long as the diagnostic system used to provide the diagnostic services has network access.

Additionally or alternatively, diagnostic system 300 may transmit data representative of the linking of the activation code to the implant ID to the cochlear implant for storage within the cochlear implant. A diagnostic system (e.g., diagnostic system 300 and/or any other diagnostic system) may subsequently determined that the cochlear implant has a valid activation code associated there with by receiving data representative of the activation code directly from the cochlear implant.

In some examples, diagnostic system 300 may store usage data together with data representative of the linking of the activation code to the implant ID. For example, after features of the diagnostic application are enabled in response to a valid activation code being provided for the cochlear implant, diagnostic system 300 may store usage data representative of one or more results of one or more tests performed with respect to cochlear implant using the enabled features of the diagnostic application. This usage data may be stored together with data representative of the linking of the activation code to the implant ID. In this manner, when the cochlear implant is subsequently connected to a diagnostic system (e.g., diagnostic system 300 or any other diagnostic system), the usage data may be available to a user of the diagnostic system.

In some examples, diagnostic system 300 may generate a report that includes a scannable indicator that the activation code is linked to the implant ID associated with the cochlear implant. The report may be in any suitable format. For example, the report may include a document (e.g., a PDF document) that may be viewed on a display screen and/or printed. The scannable indicator may be configured to facilitate enablement of the feature by an additional diagnostic system while the additional diagnostic system is communicatively coupled to the cochlear implant. To illustrate, the report may be printed by diagnostic system 300 upon completion of one or more diagnostic operations. The recipient of the cochlear implant may take the printed report to a different clinic. A diagnostic system used by the different clinic may use a barcode scanner to scan the report and thereby determine that the cochlear implant is already associated with a valid activation code.

In some examples, diagnostic system 300 may maintain log data (e.g., a usage log) associated with an activation code. For example, data representative of one or more operations performed by diagnostic system 300 using an activated feature of a diagnostic application may be stored together with data linking the activation code with an implant ID of a cochlear implant. This log data may be used to subsequently (e.g., by another diagnostic system) determine what diagnostic operations have been performed with respect to the cochlear implant. The log data may also be used to identify particular services and/or products that may be used by the recipient. For example, the log data may indicate that the recipient has a certain amount of residual hearing that would qualify him or her for a bimodal cochlear implant system configured to provide both acoustic and electric stimulation.

The systems and methods described herein may additionally or alternatively facilitate usage tracking of one or more features of a diagnostic application. For example, the systems and methods may allow a diagnostic system to keep a usage log of how many times a particular feature within a diagnostic application is used for a particular cochlear implant. The usage log may be used to perform analytics, notify a clinician of operations performed with respect to a particular recipient's cochlear implant during surgery, track usage for licensing purposes, etc.

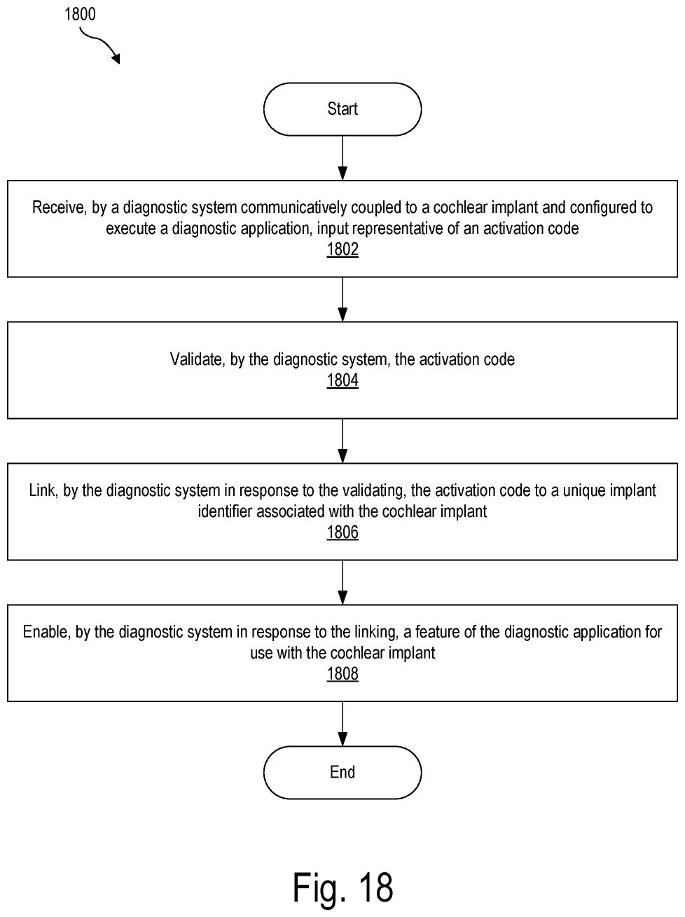

FIG. 18 illustrates an exemplary method 1800. The operations shown in FIG. 18 may be performed by diagnostic system 300 and/or any implementation thereof. While FIG. 18 illustrates exemplary operations according to one embodiment, other embodiments may omit, add to, reorder, and/or modify any of the operations shown in FIG. 18.

In operation 1802, a diagnostic system communicatively coupled to a cochlear implant and configured to execute a diagnostic application receives input representative of an activation code. Operation 1802 may be performed in any of the ways described herein.

In operation 1804, the diagnostic system validates the activation code. Operation 1804 may be performed in any of the ways described herein.

In operation 1806, the diagnostic system links, in response to the validating, the activation code to a unique implant identifier associated with the cochlear implant. Operation 1806 may be performed in any of the ways described herein.

In operation 1808, the diagnostic system enables, in response to the linking, a feature of the diagnostic application for use with the cochlear implant. Operation 1808 may be performed in any of the ways described herein.

In some examples, a non-transitory computer-readable medium storing computer-readable instructions may be provided in accordance with the principles described herein. The instructions, when executed by a processor of a computing device, may direct the processor and/or computing device to perform one or more operations, including one or more of the operations described herein. Such instructions may be stored and/or transmitted using any of a variety of known computer-readable media.

A non-transitory computer-readable medium as referred to herein may include any non-transitory storage medium that participates in providing data (e.g., instructions) that may be read and/or executed by a computing device (e.g., by a processor of a computing device). For example, a non-transitory computer-readable medium may include, but is not limited to, any combination of non-volatile storage media and/or volatile storage media. Exemplary non-volatile storage media include, but are not limited to, read-only memory, flash memory, a solid-state drive, a magnetic storage device (e.g. a hard disk, a floppy disk, magnetic tape, etc.), ferroelectric random-access memory (“RAM”), and an optical disc (e.g., a compact disc, a digital video disc, a Blu-ray disc, etc.). Exemplary volatile storage media include, but are not limited to, RAM (e.g., dynamic RAM).

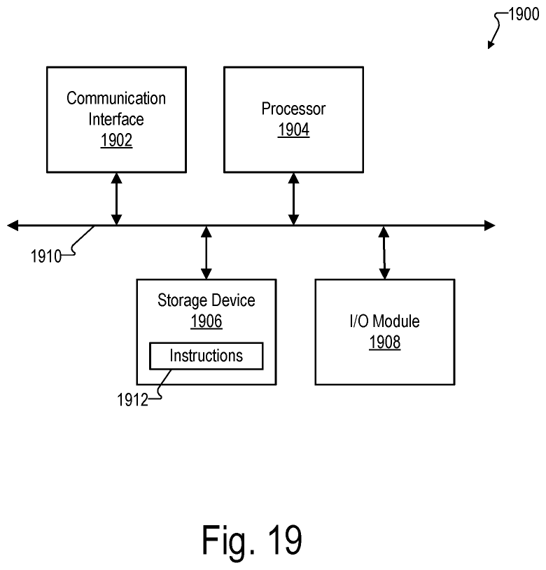

FIG. 19 illustrates an exemplary computing device 1900 that may be specifically configured to perform one or more of the processes described herein. As shown in FIG. 19, computing device 1900 may include a communication interface 1902, a processor 1904, a storage device 1906, and an input/output (“I/O”) module 1908 communicatively connected one to another via a communication infrastructure 1910. While an exemplary computing device 1900 is shown in FIG. 19, the components illustrated in FIG. 19 are not intended to be limiting. Additional or alternative components may be used in other embodiments. Components of computing device 1900 shown in FIG. 19 will now be described in additional detail.

Communication interface 1902 may be configured to communicate with one or more computing devices. Examples of communication interface 1902 include, without limitation, a wired network interface (such as a network interface card), a wireless network interface (such as a wireless network interface card), a modem, an audio/video connection, and any other suitable interface.

Processor 1904 generally represents any type or form of processing unit capable of processing data and/or interpreting, executing, and/or directing execution of one or more of the instructions, processes, and/or operations described herein. Processor 1904 may perform operations by executing computer-executable instructions 1912 (e.g., an application, software, code, and/or other executable data instance) stored in storage device 1906.

Storage device 1906 may include one or more data storage media, devices, or configurations and may employ any type, form, and combination of data storage media and/or device. For example, storage device 1906 may include, but is not limited to, any combination of the non-volatile media and/or volatile media described herein. Electronic data, including data described herein, may be temporarily and/or permanently stored in storage device 1906. For example, data representative of computer-executable instructions 1912 configured to direct processor 1904 to perform any of the operations described herein may be stored within storage device 1906. In some examples, data may be arranged in one or more databases residing within storage device 1906.

I/O module 1908 may include one or more I/O modules configured to receive user input and provide user output. I/O module 1908 may include any hardware, firmware, software, or combination thereof supportive of input and output capabilities. For example, I/O module 1908 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1908 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1908 is configured to provide graphical data to a display for presentation to a user. The graphical data may be representative of one or more graphical user interfaces and/or any other graphical content as may serve a particular implementation.

In some examples, any of the systems, computing devices, and/or other components described herein may be implemented by computing device 1900. For example, storage facility 302 may be implemented by storage device 1906, and processing facility 304 may be implemented by processor 1904.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A diagnostic system comprising:
a memory storing data representative of a diagnostic application;
a processor communicatively coupled to the memory and configured to:
execute the diagnostic application;
communicatively couple to a cochlear implant;
receive, while communicatively coupled to the cochlear implant, input representative of an activation code;
validate the activation code;
link, in response to the validation, the activation code to a unique implant identifier associated with the cochlear implant; and
enable, in response to the linking, a feature of the diagnostic application for use with the cochlear implant.

2. The diagnostic system of claim 1, wherein the feature comprises monitoring an evoked response signal recorded by an electrode disposed on an electrode lead communicatively coupled to the cochlear implant.

3. The diagnostic system of claim 1, wherein the communicative coupling to the cochlear implant comprises establishing a connection with the cochlear implant by way of a cable that connects the diagnostic system to a sound processor that is wirelessly connected to the cochlear implant by way of a coil included in a headpiece.

4. The diagnostic system of claim 1, wherein:
the activation code is included in a scannable label disposed on packaging off a surgical kit configured to be used during a surgical procedure on a recipient of the cochlear implant; and
the receiving of the input representative of the activation code comprises scanning the scannable label with a barcode scanner included in the diagnostic system.

5. The diagnostic system of claim 1, wherein:
the processor is further configured to direct a display device to display a graphical user interface associated with the diagnostic application; and
the receiving of the input representative of the activation code comprises receiving, by way of the graphical user interface, user input representative of the activation code.

6. The diagnostic system of claim 1, wherein the validating of the activation code comprises determining that the activation code has one or more valid characteristics.

7. The diagnostic system of claim 1, wherein the validating of the activation code comprises determining that the activation code is included in a database of valid activation codes.

8. The diagnostic system of claim 1, wherein the linking of the activation code to the unique implant identifier associated with the cochlear implant comprises associating the activation code with the unique implant identifier within a database.

9. The diagnostic system of claim 1, wherein the processor is further configured to store, within a storage device of the diagnostic system, data representative of the linking of the activation code to the unique implant identifier associated with the cochlear implant.

10. The diagnostic system of claim 1, wherein the processor is further configured to transmit, to a computing device by way of a network for remote storage by the computing device, data representative of the linking of the activation code to the unique implant identifier associated with the cochlear implant.

11. The diagnostic system of claim 1, wherein the processor is further configured to transmit, to the cochlear implant for storage within the cochlear implant, data representative of the linking of the activation code to the unique implant identifier associated with the cochlear implant.

12. The diagnostic system of claim 1, wherein the processor is further configured to generate a report that includes a scannable indicator that the activation code is linked to the unique implant identifier associated with the cochlear implant, the scannable indicator configured to facilitate enablement of the feature by an additional diagnostic system for use with the cochlear implant while the additional diagnostic system is communicatively coupled to the cochlear implant.

13. The diagnostic system of claim 1, wherein the processor is further configured to:
query, prior to receiving the input representative of the activation code, the cochlear implant for the unique implant identifier; and
receive, in response to the query, the unique implant identifier from the cochlear implant.

14. The diagnostic system of claim 1, wherein the processor is further configured to:
communicatively decouple from the cochlear implant;
communicatively couple to an additional cochlear implant;
receive, while communicatively coupled to the additional cochlear implant, input representative of an additional activation code;
determine that the additional activation code is invalid;
disable, in response to the determination that the additional activation code is invalid, the feature of the diagnostic application for the additional cochlear implant.

15. The diagnostic system of claim 1, wherein the processor is further configured to maintain an additional feature of the diagnostic application as being disabled for use with the cochlear implant.

16. A diagnostic system comprising:
a computing module comprising:
a display screen, and
a processor; and
a base module configured to attach to the computing module and serve as a stand for the computing module, the base module housing an interface unit configured to be communicatively coupled to the processor and to a cochlear implant while the base module is attached to the computing module;
wherein the processor is configured to:
execute a diagnostic application;
direct the display screen to display a graphical user interface associated with the diagnostic application;
query, by way of the interface unit, the cochlear implant for a unique implant identifier associated with the cochlear implant;
receive, in response to the query, the unique implant identifier from the cochlear implant;
receive, while the interface unit is communicatively coupled to the cochlear implant, input representative of an activation code;
validate the activation code;
link, in response to the validation, the activation code to the unique implant identifier associated with the cochlear implant;
enable, in response to the linking, a feature of the diagnostic application for use with the cochlear implant; and
indicate, within the graphical user interface, that the feature is enabled for use with the cochlear implant.

17. A method comprising:
receiving, by a diagnostic system communicatively coupled to a cochlear implant and configured to execute a diagnostic application, input representative of an activation code;
validating, by the diagnostic system, the activation code;
linking, by the diagnostic system in response to the validating, the activation code to a unique implant identifier associated with the cochlear implant; and
enabling, by the diagnostic system in response to the linking, a feature of the diagnostic application for use with the cochlear implant.

18. The method system of claim 17, further comprising storing, by the diagnostic system within a storage device of the diagnostic system, data representative of the linking of the activation code to the unique implant identifier associated with the cochlear implant.

19. The method system of claim 17, further comprising transmitting, to a computing device by way of a network for remote storage by the computing device, data representative of the linking of the activation code to the unique implant identifier associated with the cochlear implant.

20. The method system of claim 17, further comprising transmitting, to the cochlear implant for storage within the cochlear implant, data representative of the linking of the activation code to the unique implant identifier associated with the cochlear implant.
